(19) United States
(12) Patent Application Publication
(10) Pub. No.: US 2024/0148515 A1
(43) Pub. Date: May 9, 2024

(54) INTERSPINOUS PROCESS FIXATION DEVICE

(71) Applicant: BFM Holdings, LLC, Austin, TX (US)

(72) Inventors: Randall F. Dryer, Austin, TX (US); Ali Kiapour, Newton, MA (US); Liam Joseph Raymond McKay, Boston, MA (US)

(21) Appl. No.: 18/502,593

(22) Filed: Nov. 6, 2023

Related U.S. Application Data
(60) Provisional application No. 63/382,423, filed on Nov. 4, 2022.

(51) Int. Cl. A61F 2/44; A61B 17/86; A61F 2/30

(52) U.S. Cl. CPC A61F 2/4405; A61B 17/8625; A61F 2/30749; A61F 2002/2835; A61F 2002/3055; A61F 2002/3092; A61F 2002/3093

(57) ABSTRACT

A spinous process fixation device is provided. The spinous process device may include a transverse bridge member having a width that is perpendicular to a longitudinal axis, a first side plate that may be positioned at a first end of the transverse bridge and having at least one first grip projection, and a second side plate that may be positioned at a second end of the transverse bridge member and having at least one second grip projection. The first grip projection and the one second grip projection may each protrude towards the transverse bridge member.

### CROSS-REFERENCE TO RELATED APPLICATION

This application claims priority to U.S. Patent Application Ser. No. 63/382,423 filed Nov. 4, 2022, entitled, “Interspinous Process Fixation Device” which is incorporated herein by reference in its entirety.

### FIELD OF THE INVENTION

The present disclosure relates generally to spinal devices, and more particularly interspinous process (“ISP”) fixation devices with improved flexural and bone grafting functionalities.

### BACKGROUND

There are a number of needs in the art related to the features and functionalities of existing interspinous process fixation devices. Each vertebra in the spine includes bone protrusions arising from the posterior side of the bone called spinous processes. Damage to the spine may occur for a variety of reasons such as injury or illness. Severe or debilitating pain can result from such damage. In some instances, artificial assistance may be necessary to address such damage.

There are many types of hardware for the fixation of spinous processes to stabilize the spine. The majority of current interspinous process fixation implants are solid bodied, often with few machined cavities for increased bone growth through the implant and shorter fixation times. Solely having slot style cavities in a solid body implant may limit strain within the implant. This may minimize the promotion of bone growth on the hardware. Further, the solid body of the implant may block, inhibit, and/or delay bone through-growth in all areas except for the designed cavities. These slot style cavities through the solid body must typically be small enough to maintain the strength of the implant, which may also limit the volume of bone graft which may be packed into it if the surgeon elects to do so prior to implantation.

Another limitation of existing ISP fixation technology is the rigidity of the implant construct that comes in contact with the bone. The rigid implant may result in stress shielding and improper load distribution at the bone-implant interface which may result in fracture of the bone or lack of development of fusion at the fixation site. As such, there is a long felt but unresolved need for an improved interspinous process fixation device which may improve bone grafting, anchorage, and bone growth.

### SUMMARY

Provided herein are several embodiments of a spinous process fixation device and bone screw designed to overcome many of the shortcomings and limitations of the prior solutions discussed above. Generally, the spinous process fixation device and screw provided herein may be implanted, inserted, incorporated, and/or otherwise applied to a body at a fixation site. The spinous process fixation device may be designed to proliferate bone growth and permit and limit.

According to a first aspect, a spinous process fixation device comprising a transverse bridge member, a first side plate, and a second side plate is provided. The transverse bridge member may have a width that is perpendicular to a longitudinal axis of the spinous process fixation device. The first side plate may be positioned about a first end of the transverse bridge member, and the second side plate may be positioned about a second end of the transverse bridge member. Each the first side plate and the second side plate may comprise at least one grip projection. A first grip projection of the first side plate may protrude towards the transverse bridge member, and a second grip projection of the second plate may protrude towards the transverse bridge member. In some embodiments, the grip projections may be configured to contact and engage with the surrounding environment at the site of installation (i.e., the fixation site). The grip projections may function to maintain position of the spinous process fixation device and the components therein (e.g., the first side plate, the second side plate, and the transverse bridge member).

In some embodiments, the spinous process fixation device may further comprise a fixation member and a screw. The fixation member and the screw may be used to adjust a distance between the first side plate and the second side plate along the transverse bridge member. For example, the fixation member and the screw may be used to narrow or widen a distance between the first and second side plates. In other embodiments, the spinous process fixation device may comprise a guide channel.

In yet other embodiments, at least one of the transverse bridge member, the first side plate, and the second side plate may comprise a plurality of open lattice structures. In such embodiments, the plurality of open lattice structures may define a plurality of openings that may allow for bone growth through the spinous process fixation device (i.e., bone through-growth). Further, the bone may not only grow through the spinous process fixation device but may also grow through the spinous process fixation device in more than one direction. In other words, the open lattice structures, and the openings therein, may allow for multidirectional bone growth. In some embodiments, a plurality of openings about the transverse bridge member may be larger than a plurality of openings about the first and second side plates.

In at least one embodiment, the first side plate and the second side plate may be oriented perpendicularly with respect to the longitudinal axis. As such, the first side plate and the second side plate may be oriented parallelly with respect to each other.

In other embodiments, the first side plate and the second side plate may be oriented at an angle with respect to the longitudinal axis.

According to an additional embodiment, a spinous process fixation device comprising a spring member, a first side plate, and a second side plate is provided. The spring member may have a diameter that is perpendicular to a longitudinal axis of the spinous process fixation device. The first side plate may be positioned about a first end of the spring member, and the second side plate may be positioned at about a second end of the spring member. Each the first side plate and the second side plate may comprise at least one grip projection. A first grip projection of the first side plate may protrude towards the spring member, and, similarly, a second grip projection of the second plate may protrude towards the spring member. In some embodiments, the spring member may be configured to limit lateral flexure.

In other embodiments, the first side plate and the second side plate may be oriented perpendicularly with respect to the longitudinal axis. In such embodiments, the first and second side plates may be oriented parallelly with respect to each other.

In yet other embodiments, the spring member may comprise a spring coil. In such embodiments, the spring member may be configured to compress or decompress to adjust a distance between the first side plate and the second side plate.

In some embodiments, at least one of the first side plate and the second side plate may comprise a solid member. In other embodiments, the spinous process fixation device may comprise a biocompatible material such as a Grade V titanium material.

According to yet another embodiment, a surgical screw comprising at least one helical thread and at least one inner framework helix is provided. The at least one helical thread may be arranged in either a right-hand helix or a left-hand helix. Further, the at least one helical thread may have a first thickness that may be defined by an inner diameter. The at least one inner framework helix may be arranged in the other of the right-hand helix or the left-hand helix and may have a second thickness defined by an outer diameter. The inner diameter may be smaller than the outer diameter, thereby creating an overlap of the first and second thicknesses. In some embodiments, the overlap may be between about 25%-75% of the first thickness of the at least one helical thread. In other embodiments, the at least one inner framework helix may be rough, wherein the rough surface may comprise a plurality of raises and depressions. In yet other embodiments, the surface of the at least one helical thread may be smooth.

These and other aspects and advantages of the present embodiments will become apparent to those skilled in the art after considering the following detailed description in connection with the accompanying drawings.

### BRIEF DESCRIPTION OF THE DRAWINGS

FIG. 1 is a perspective view of one embodiment of an interspinous process fixation device in accordance with the teachings of the present disclosure;

FIG. 2 is a top plan view of the interspinous process fixation device from FIG. 1;

FIG. 3 is a bottom plan view of the interspinous process fixation device from FIG. 1;

FIG. 4 is a front elevation view of the interspinous process fixation device from FIG. 1;

FIG. 5 is a rear elevation view of the interspinous process fixation device from FIG. 1;

FIG. 6 is a first side elevation view of the interspinous process fixation device from FIG. 1;

FIG. 7 is a second side elevation view of the interspinous process fixation device from FIG. 1;

FIG. 8 is a perspective view of a second embodiment of an interspinous process fixation device in accordance with the teachings of the present disclosure;

FIG. 9 is a top plan view of the interspinous process fixation device from FIG. 8;

FIG. 10A is a perspective view of one embodiment of an interspinous process fixation device inserted into a spine in accordance with the teachings of the present disclosure;

FIG. 10B is a side perspective view of the interspinous process fixation device from FIG. 10A;

FIG. 10C is a front perspective view of the interspinous process fixation device from FIG. 10A;

FIG. 11 is a perspective view of one embodiment of a double-helix screw in accordance with the teachings of the present disclosure;

FIG. 12 is a first side perspective view of the one embodiment of the double-helix screw from FIG. 11;

FIG. 13 is a second side perspective view of the one embodiment of the double-helix screw from FIG. 11;

FIG. 14 is a top plan view of the one embodiment of the double-helix screw from FIG. 11;

FIG. 15 is a bottom plan view of the one embodiment of the double-helix screw from FIG. 11;

FIG. 16 is a perspective view of a third embodiment of an interspinous process fixation device in accordance with the teachings of the present disclosure;

FIG. 17 is a rear elevation view of the interspinous process fixation from FIG. 16;

FIG. 18 is a top plan view of the interspinous process fixation device from FIG. 16;

FIG. 19 is a bottom plan view of the interspinous process fixation device from FIG. 16;

FIG. 20 is a first side elevation view of the interspinous process fixation device from FIG. 16;

FIG. 21 is a second side elevation view of the interspinous process fixation device from FIG. 16;

FIG. 22 is a perspective view of an embodiment of an interspinous process fixation device filled with a bone graft material.

### DETAILED DESCRIPTION

Before any embodiments are described in detail, it is to be understood that the disclosure is not limited in its application to the details of construction and the arrangement of components set forth in the following description or illustrated in the following drawings, which is limited only by the claims that follow the present disclosure. The disclosure is capable of other embodiments, and of being practiced, or of being carried out, in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “connected,” and “coupled,” and variations thereof are used broadly and encompass both direct and indirect connections and couplings. Further, “connected” and “coupled” are not restricted to physical or mechanical connections or couplings.

The following description is presented to enable a person skilled in the art to make and use embodiments of the disclosure. Various modifications to the illustrated embodiments will be readily apparent to those skilled in the art, and the generic principles herein can be applied to other embodiments and applications without departing from embodiments of the disclosure. Thus, embodiments of the disclosure are not intended to be limited to embodiments shown but are to be accorded the widest scope consistent with the principles and features disclosed herein. The following detailed description is to be read with reference to the figures, in which like elements in different figures have like reference numerals. Skilled artisans will recognize the examples provided herein have many useful alternatives and fall within the scope of embodiments of the disclosure.

Additionally, while the following discussion may describe features associated with specific devices or embodiments, it is understood that additional devices, systems, and/or features can be used with the described device and methods and that the discussed devices and features are used to provide examples of possible embodiments, without being limited.

The present disclosure is directed toward an ISP fixation device that may be implanted, inserted, or otherwise incorporated into a body (e.g., a human body) to facilitate and/or promote bone growth. In one example, the ISP device may be incorporated in bony areas such as the spinal column of a human body. The ISP device may contact spinal anatomy and, in some embodiments, may limit movement. The ISP device may be incorporated in other areas of a body and is not particularly limited to the spinal column.

Also provided herein are embodiments of a bone screw that, in some embodiments, may be used in conjunction with the above-described ISP device. Generally, the bone screw may be coupled, incorporated, connected, or otherwise used with the ISP device to functionally provide fixation or security between components of the ISP device. Further, the bone screw may be coupled, incorporated, connected, or otherwise used with the ISP device to functionally provide fixation or security to the surrounding environment and/or area of installation within the body. Yet further, the bone screw may promote bone growth through the screw.

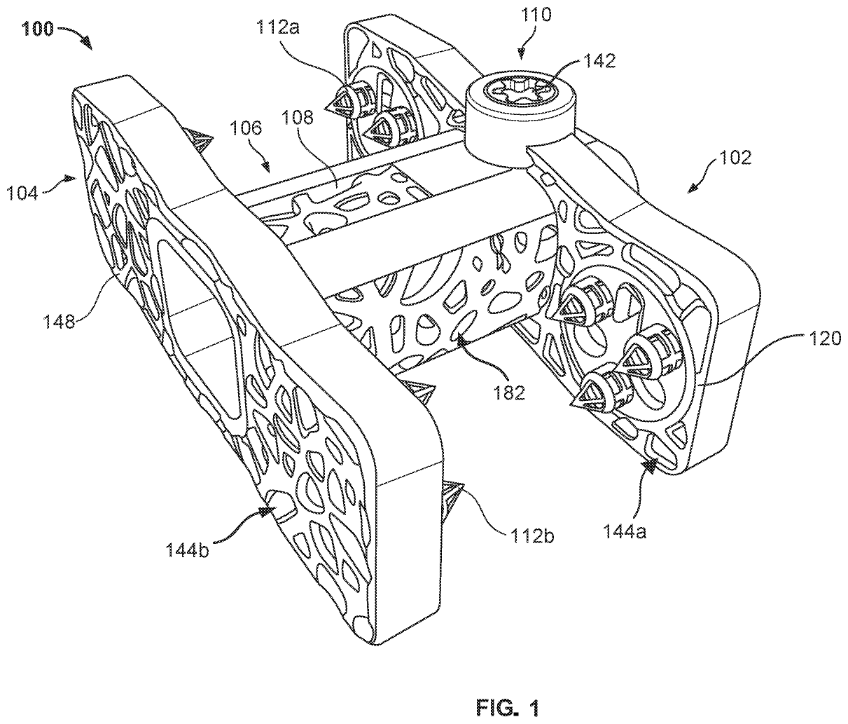

FIG. 1 illustrates an embodiment of an ISP device 100 comprising a first side plate 102, a second side plate 104, and a transverse bridge member 106. The ISP device 100 may further comprise at least one guide channel 108, at least one fixation member 110, and one or more grip projections 112. In preferred embodiments, the ISP device 100 may comprise a plurality of grip projections 112. Generally, the shape of the first side plate 102 and the second side plate 104 may both be, or substantially similar to, a rectangular prism. In alternative embodiments, the first side plate 102 and the second side plate 104 may each have a three-dimensional shape that may include angular edges, rounded edges, or a combination thereof. Further, in some embodiments, the shape of the first side plate 102 may be different than the shape of the second side plate 104. For example, the first side plate 102 may have a shape that may be, or substantially similar to, a rectangular prism, and the second side plate 104 may have a shape that may be, or substantially similar to, a cube.

In some embodiments, the first side plate 102 and the second side plate 104 may be substantially parallel to each other. Alternatively, the first side plate 102 and the second side plate 104 may be oriented at an angle. The first side plate 102 may be disposed about a transverse bridge member first end 114, and the second side plate 104 may be disposed about a transverse bridge member second end 116. The transverse bridge member 106 may extend between and intersect the first side plate 102 and the second side plate 104. For example, the transverse bridge member 106 may be positioned in the center of the ISP device 100 and may intersect the first side plate 102 and the second side plate 104 such that the first side plate 102 and second side plate 104 are bisected by the transverse bridge member 106. In other embodiments, the transverse bridge member 106 may be positioned such that it intersects the first side plate 102 and the second side plate 104 and may create unequal portions.

In various embodiments, the transverse bridge member 10 may intersect the first side plate 102 and the second side plate 104 about perpendicularly (i.e., 90°). In other embodiments, the transverse bridge member 106 may intersect the first side plate 102 and the second side plate 104 at an angle between about 60 degrees and 90 degrees. Alternatively, the transverse bridge member 106 may intersect the first side plate 102 and the second side plate 104 at an angle that is more or less than about 60 degrees and 90 degrees. Preferably, the transverse bridge member 106 may have a width and a longitudinal axis that may be described as being perpendicular to the width of the transverse bridge member 106. The first and second side plates 102 and 104 may be parallel and disposed on opposite ends of the transverse bridge member 106 along the longitudinal axis.

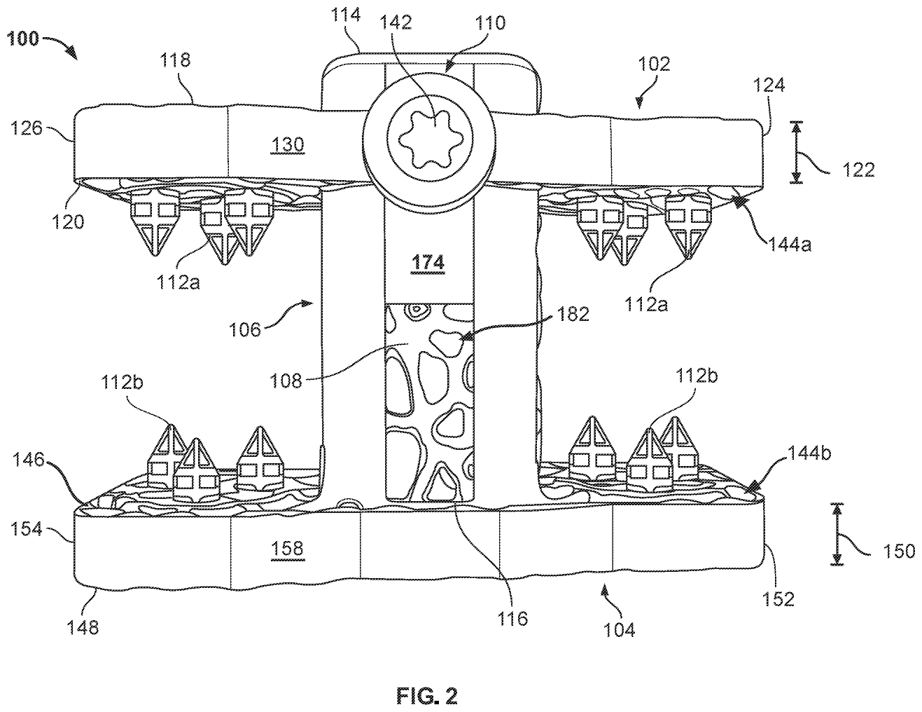

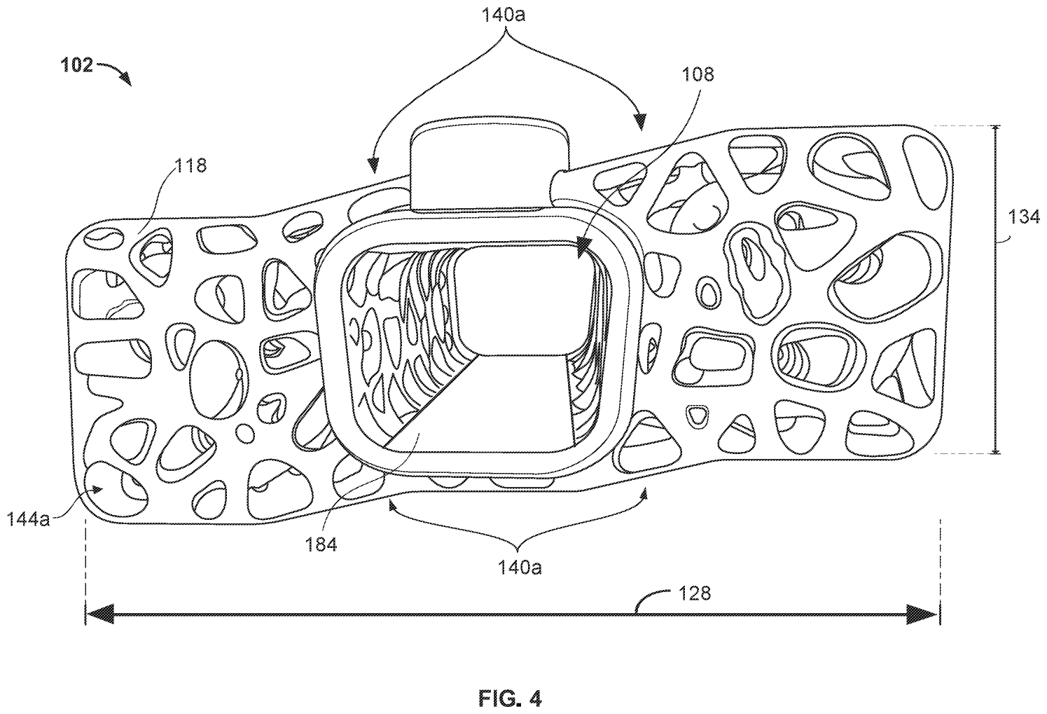

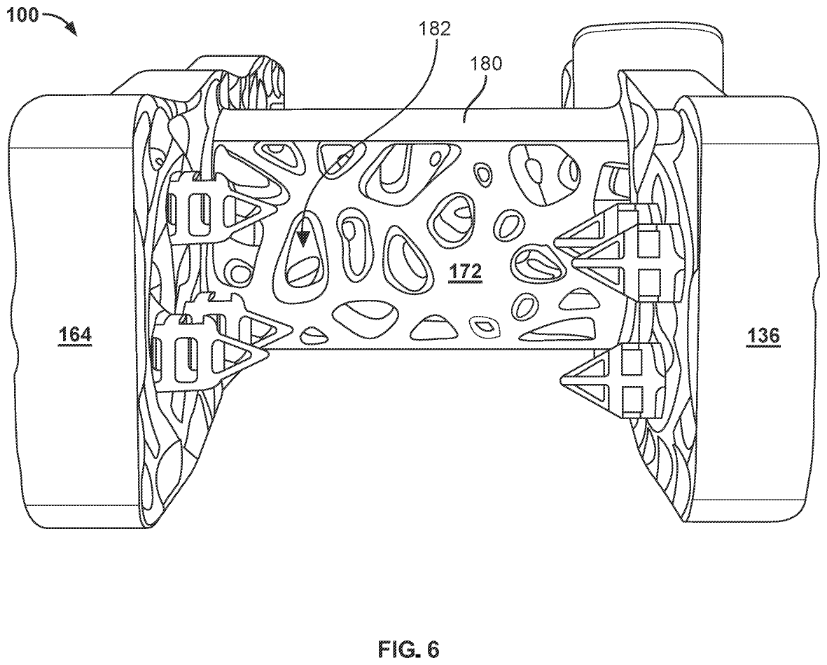

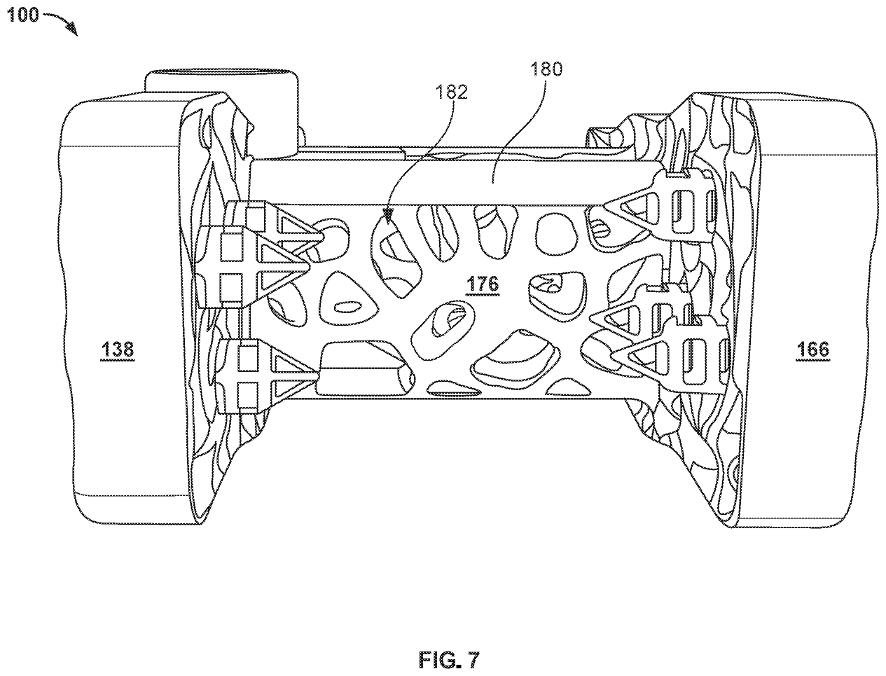

Referring to FIGS. 2-7, the first side plate 102 may include a first side plate first side 118, and a first side plate second side 120. A distance between the first side plate first side 118 and the first side plate second side 120 may define a thickness 122, as illustrated in FIG. 2. Moreover, the first side plate 102 may include a first side plate first end 124 and a first side plate second end 126. A distance from the first side plate first end 124 and the first side plate second end 126 may define a length 128 dimension, as best shown in FIG. 4. The first side plate 102 may include a first side plate top face 130 and a first side plate bottom face 132. A distance from the first side plate top face 130 and the first side plate bottom face 132 may define a width 134. The first side plate 102 may include a first side plate first end surface 136 and a first side plate second end surface 138. The first side plate first end surface 136 and the first side plate second end surface 138 may have the thickness 122 and the width 134 dimensions. Further, the first side plate 102 may also have at least one angular step 140a along the first side plate top face 130 and the first side plate bottom face 132.

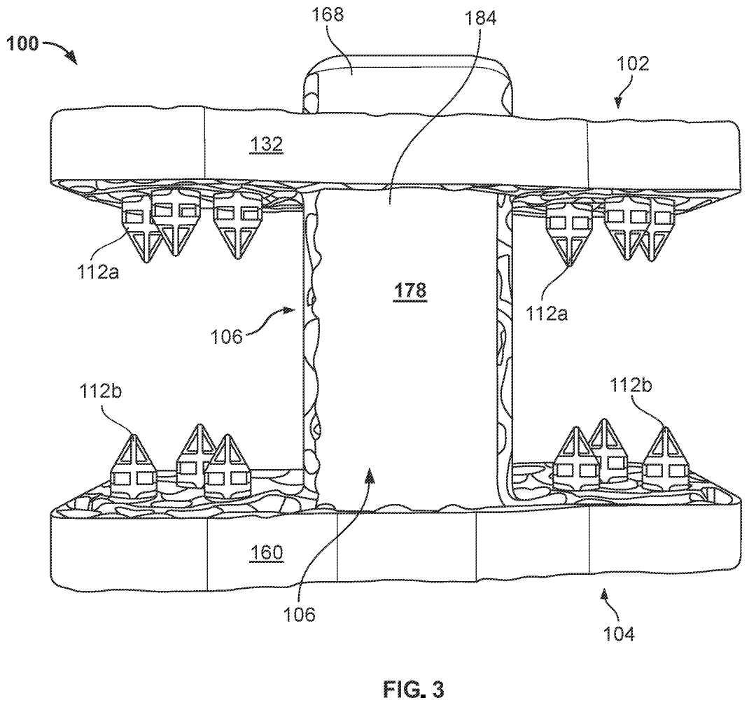

The first side plate second side 120 may include one or more protruding grip projections 112a. The one or more grip projections 112a may be configured to contact and/or engage with the surrounding environment of the ISP device 100 such that the ISP device 100 may be positioned and maintained in place. The one or more grip projections 112a may be, for example, conical or pyramidal. Alternatively, the one or more grip projections 112a may have a shape that is angular, rounded, or a combination thereof. The one or more grip projections 112a may have a base end that extends from the first side plate second side 120 to a distal end. In one non-limiting example, the grip projections 112a may have a conical shape with a rounded base end and a pointed tip (distal end). Further, the one or more grip projections 112a may protrude inwardly with respect to the second side plate 104 and the transverse bridge member 106. That is, the one or more grip projections 112a may protrude towards the second side plate 104 and the transverse bridge member 106, as shown best in FIGS. 2 and 3. In preferred embodiments, the first side plate second side 120 may include one or more grip projections 112a on either side of the transverse bridge member 106.

For example, as illustrated in FIG. 2, the first side plate second side 120 may include a group of three grip projections 112a that are positioned about the first side plate second side 120 on one side of the transverse bridge member 106, and, on the other side of the transverse bridge member 106, another group of about three grip projections 112a may be positioned about the first side plate second side 120. Alternatively, the number of grip projections 112a on either side of the transverse bridge member 106 may be different such that there are more grip projections 112a on one side of the transverse bridge member 106 than the other side.

The fixation member 110 of the first side plate 102 may have a rounded periphery. Alternatively, the fixation member 110 may have a periphery that is angular or a combination of rounded and angular. The fixation member 110 may be configured to secure the ISP device 100 in a desired position, orientation, and/or location. In some embodiments, the fixation member 110 may include a set screw 142. The set screw 142 may be configured to receive a Torx driver head. Alternatively, other drivers such as square, Phillips, hex, slot, or any other driver now known or hereafter developed, may be utilized.

A distance between the first side plate 102 and the second side plate 104 along the transverse bridge member 106 may be adjusted by the fixation member 110 and the set screw 142. For example, at the fixation site for the ISP device 100, the fixation member 110 and the set screw 142 may be used to narrow or broaden the distance between the first side plate 102 and the second side plate 104 along the transverse bridge member 106 until a desirable distance is reached.

The first side plate 102 may comprise a first side plate porous member 144a, as best shown in FIG. 4. The first side plate porous member 144a may include an open lattice structure that may create one or more openings that may be designed to promote, facilitate, or otherwise encourage bone growth through the ISP device 100. In various embodiments, the first side plate 102 may comprise a plurality of first side plate porous members 144a that promotes contiguous bone growth through the ISP device 100.

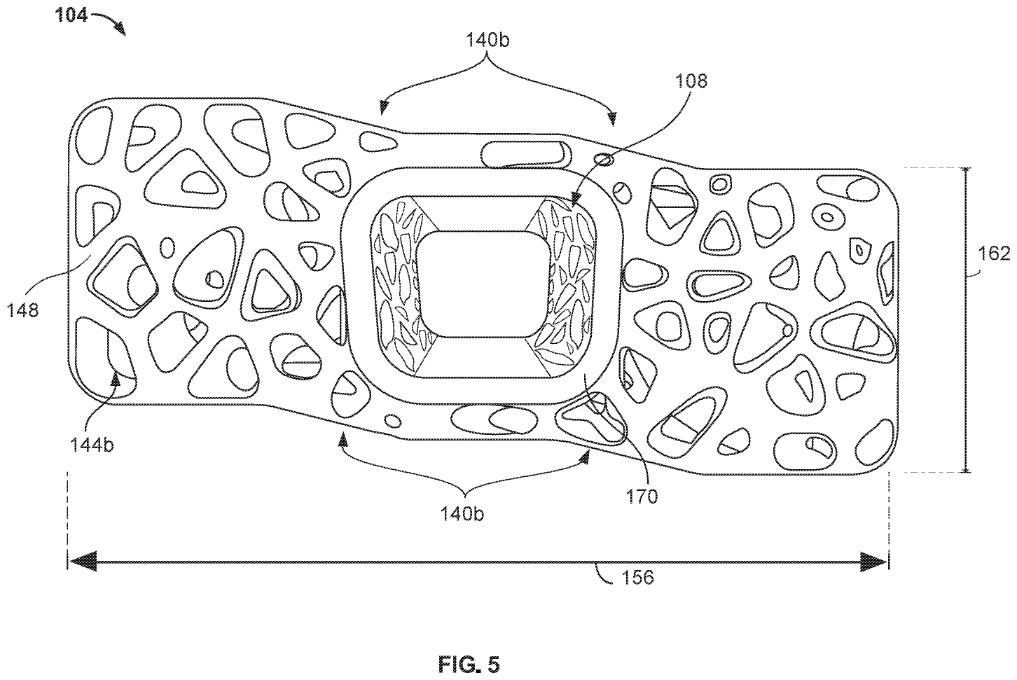

The second side plate 104 may include a second side plate first side 146 and a second side plate second side 148. A distance between the second side plate first side 146 and the second side plate second side 148 may define a thickness 150, as best shown in FIG. 2. Moreover, the second side plate 104 may include a second side plate first end 152 and a second side plate second end 154. A distance from the second side plate first end 152 and the second side plate second end 154 may define a length 156 dimension, as best shown in FIG. 5. The second side plate 104 may include a second side plate top face 158 and a second side plate bottom face 160. A distance from the second side plate top face 158 and the second side plate bottom face 160 may define a width 162. The second side plate 104 may include a second side plate first end surface 164 and a second side plate second end surface 166. The second side plate first end surface 164 and the second side plate second end surface 166 may also have the thickness 150 and the width 162 dimensions, as best shown in FIGS. 2, 5, and 6. Further, the second side plate 104 may have at least one angular step 140b along the second side plate top face 158 and the second side plate bottom face 160.

The second side plate first side 146 may include one or more grip projections 112b. The one or more grip projections 112b of the second side plate first side 146 may be substantially similar or identical to the one or more grip projections 112a of the first side plate second side 120. The one or more grip projections 112b may be configured to contact and/or engage with the surrounding environment of the ISP device 100 such that the ISP device 100 may be positioned and maintained in place amongst other functionalities Like the one or more grip projections 112a, the one or more grip projections 112b may have a 3D shape that is conical or pyramidal. Alternatively, the one or more grip projections 112b may have a shape that is angular, rounded, or a combination thereof. In exemplary embodiments, the one or more grip projections 112b may have a base end that extends from the second side plate first side 146 to a distal end. In one non-limiting example, the grip projection 112b has a conical shape with a rounded base end and a pointed tip (distal end). Further, the one or more grip projections 112b may protrude inwardly with respect to the first side plate 102 and the transverse bridge member 106. That is, the one or more grip projections 112b may protrude towards the first side plate 102 and the transverse bridge member 106, as shown best in FIGS. 2 and 3.

In some embodiments, the second side plate first side 146 may include one or more grip projections 112b on each side of the transverse bridge member 106. For example, the second side plate first side 146 may include a group of about three grip projections 112b that are positioned about the second side plate first side 146 on one side of the transverse bridge member 106, and, on the other side of the transverse bridge member 106, another group of about three grip projections 112b may be positioned about the second side plate first side 146. Alternatively, the number of grip projections 112b on either side of the transverse bridge member 106 may be different such that there are more grip projections 112b on one side of the transverse bridge member 106 than the other side.

The second side plate 104 may comprise a second side plate porous member 144b. The second side plate porous member 144b may include an open lattice structure that creates one or more openings that may be designed to promote, facilitate, or otherwise encourage bone growth through the ISP device 100. In various embodiments, the second side plate 104 may comprise a plurality of second side plate porous members 50b that promotes contiguous bone growth through the ISP device 100.

The transverse bridge member 106 may include a transverse bridge member first end 114 and transverse bridge member second end 116. The transverse bridge member first end 114 may comprise a first end solid portion 168, and the transverse bridge member second end 116 may comprise a second end solid portion 170. Further, the transverse bridge member 106 may comprise a first side 172, a second side 174, a third side 176, and a fourth side 178. In various embodiments, the first side 172, the second side 174, the third side 176, and the fourth side 178 may be connected or coupled to each other by curved side transitions 180 which may form a substantially cylindrical guide channel 108. In other embodiments, the guide channel 108 have a tubular shape with rounded transitions. Alternatively, the guide channel 108 may be alternative shapes which may be angular, rounded, or a combination thereof.

In various embodiments, the transverse bridge member 106 may include pores or porous members 182. The porous members 182 may be through one or more of the sides (e.g., first side 172, the second side 174, the third side 176, and/or the fourth side 178). In some embodiments, the porous members 182 may be designed as an open lattice structure that may create openings for promoting and/or allowing for bone growth. For example, the open lattice structures may allow for bone to grow through the ISP device 100.

As best shown in at least FIGS. 2, 3, 6, and 7, the first side 172, the second side 174, and the third side 176 may include porous members 182, and the fourth side 178 may include a solid wall 184. In alternative embodiments, one or more sides of the transverse bridge member 106 may include porous members 182 and one or more sides may include one or more solid walls 184.

Generally, in one nonlimiting example of the ISP device 100 being inserted into a spine, the ISP device 100 and the components therein may be adjusted to fit between anatomy of the spine. In particular, the spine comprises vertebrates that comprise vertebrae bodies, spinous processes, and transverse processes. The ISP device 100 may be inserted between the spinous processes of two vertebrates. The ISP device 100 may then be secured in place by the fixation member 110 and the set screw 142. The fixation member 110 and set screw 142 may be used to adjust the distance between the first side plate 102 and the second side plate 104 along the transverse bridge member 106. For example, the fixation member 110 and set screw 142 may be used to narrow the distance between the first side plate 102 and the second side plate 104. Further, the fixation member 110 and set screw 142 may be used to widen the distance between the first side plate 102 and the second side plate 104. In some embodiments, the first side plate 102 and the second side plate 104 may slide along the transverse bridge member 106 until a desired distance between the first side plate 102 and the second side plate 104 is achieved and/or until the ISP device 100 is in a desired position and orientation in and along the spine 186.

In preferred embodiments, the distance may be adjusted until the first plate grip projections 112a and the second plate grip projections 112b contact the spinous processes. The first plate grip projections 112a and the second plate grip projections 112b may contact the spinous processes by engaging with the spinous processes, for example by physically touching a surface or sub-surface level of the spinous processes. In some embodiments, the first plate grip projections 112a and the second plate grip projections 112b may interdigitate with the spinous processes.

The ISP device 100 may be manufactured by 3D printing. Alternatively, the ISP device 100 may be manufactured by other processes such as casing, molding machining, joining, forging, and the like. In preferred embodiments, the ISP device 100 may be additively manufactured with a biocompatible material (for example, Grade V titanium) using selective laser melting. In other embodiments, the ISP device 100 can be made using polyethylethylketone (PEEK) plastic, nitinol metal or carbon fiber embedded additive manufacturing methods and the like. One benefit amongst others of laser melting is that laser melting may create a roughened surface, thereby increasing bone growth through (i.e., bone through-growth) the ISP device 100. In various embodiments, the ISP device 100 may promote, facilitate, or otherwise provide opportunity for bone through-growth in multiple directional planes due to the open lattice structures in at least one of the first side plate 102, the second side plate 104, and the transverse bridge member 106. Due to the ISP device 100 promoting, facilitating, or otherwise providing opportunities for the bone growth to proliferate through the ISP device 100 in multiple directions, a more distributed fusion (rather than one planar fusion) may be achieved. Another benefit of the ISP device 100 may be that the construction (e.g., a relatively rough surface, a relatively smooth surface, a plurality of porous members, open lattice structures, etc.) of the ISP device 100 also allows for a greater volume of bone graft to be packed into the ISP device 100. In one non-limiting example, the lattice structure allows for pre-packing of the bone graft, morselized bone, or biologics into the construct prior to instrumentation into the surgical site.

Moreover, the macro-porous construction of the ISP device 100 may facilitate, promote, or otherwise provide opportunity for bone through-growth. That is, bone growth may proliferate at a faster rate or with more ease through an ISP device 100 comprising a macro-porous construction in comparison to a solid body ISP device. Further, the macro-porous construction may allow for a greater volume of bone graft to be packed into the ISP device 100. For example, the open gaps created by the open lattice structure in the ISP device 100 may provide volume for the packing of a bone graft material. This may secure the pre-packed material within the ISP device 100 while allowing for relatively easier accessibility from the surrounding organic material once implanted. In some embodiments, the open lattice structure may have, form, or establish a snowshoe-like contact surface with the surrounding environment such as any adjacent bone. The open volume within the lattice may allow bone through growth but may also prevent the ISP device 100 from subsiding into the bone.

In other embodiments, the open lattice structure may also function as a fixation-promoting and biomimetic structure for cell attachment. The elimination of a solid bodied implant may reduce stress shielding on surrounding bone due to the micro strain within the ISP device, which may promote bone growth and hasten implant fixation. Due to the presence of open lattice structure in the construct, mechanical stiffness of the construct under mechanical loads may be relatively lower compared to other conventional solid ISP devices. The lower stiffness of the construct may facilitate development of the micro-strains at the surface of the ISP device 100 that may interface with bone. The micro-strain may promote the bone growth through the implant by subjecting the bone cells to mechanical strains required for cell differentiation and proliferation.

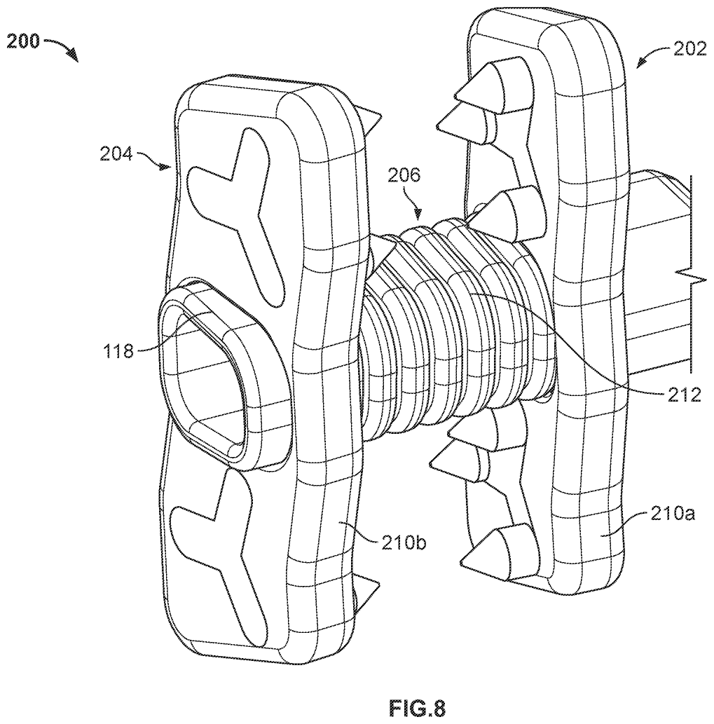

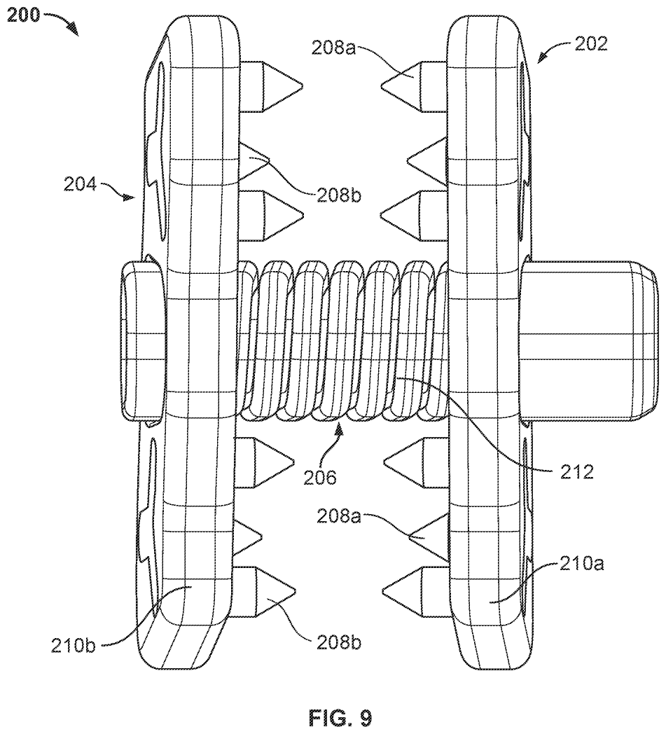

Turning to FIGS. 8 and 9, an embodiment of an alternative ISP device 200 according to the present disclosure is provided. The ISP device 200 may comprise a first side plate 202, a second side plate 204, and a mid-spring portion 206. The ISP device 200 may further comprise one or more grip projections 208. The ISP device 200, including the first side plate 202, the second side plate 204, and the one or more grip projections 208, may be substantially similar to the above-mentioned embodiment of ISP device 100. For example, the ISP device 200 may include a first end solid portion and a second end sold portion. In another example, the mid-spring portion 206 may have a diameter and a longitudinal axis that is perpendicular to the diameter of the mid-spring portion 206. The first and second side plates 202 and 204 may be parallel and disposed on opposite ends of the mid-spring portion 206 along the longitudinal axis.

However, in some embodiments, the ISP device 200 may include one or more solid members 210 rather than porous members. For example, the first side plate 202 may include a first side plate solid member 210a. Continuing this example, in some embodiments, the second side plate 204 may include a second side plate solid member 210b. Alternatively, ISP device 200 may comprise a combination of solid members 210 and porous members. Further, in some embodiments, the mid-spring portion 206 may replace the transverse bridge member. The mid-spring portion 206 may comprise a spring coil 212. In this embodiment, the second side plate 204 may not include a fixation member. Instead, the spring coil 212 may be used to compress or decompress the ISP device 200 to adjust the distance between the first side plate 202 and the second side plate 204. For example, the spring coil 212 may be configured to move the first side plate 202 and the second side plate 204 closer to, or farther away from, one another.

The midspring portion 206 and spring coil 212 may permit or limit lateral flexure between the first side plate 202 and the second side plate 204. The midspring portion 206 and spring coil 212 may adjust and/or adapt to the surrounding environment while also permitting or limiting lateral flexure. For example, an ISP device 200 implanted or inserted into a spine at the fixation site may be designed to conform to the surrounding spinal anatomy such as the width between spinous processes, and, upon installation, implantation, or otherwise insertion of the ISP device 200, the ISP device 200 may be configured to limit movement such as flexure.

The ISP device 200 may be manufactured according to the process previously described herein. In other words, the ISP device 200 may be manufactured similarly to the ISP device 100. Specifically, the ISP device 200 may be manufactured using 3D printing manufacturing methods, and may be made with Grade V titanium, PEEK (polyethylethylketone) plastic, nitinol metal, or carbon fiber embedded additive manufacturing methods. Alternatively, the ISP device 200 may be manufactured by other processes such as casing, molding machining, joining, forging, and the like. Further, the ISP device 200 may be manufactured with another biocompatible material.

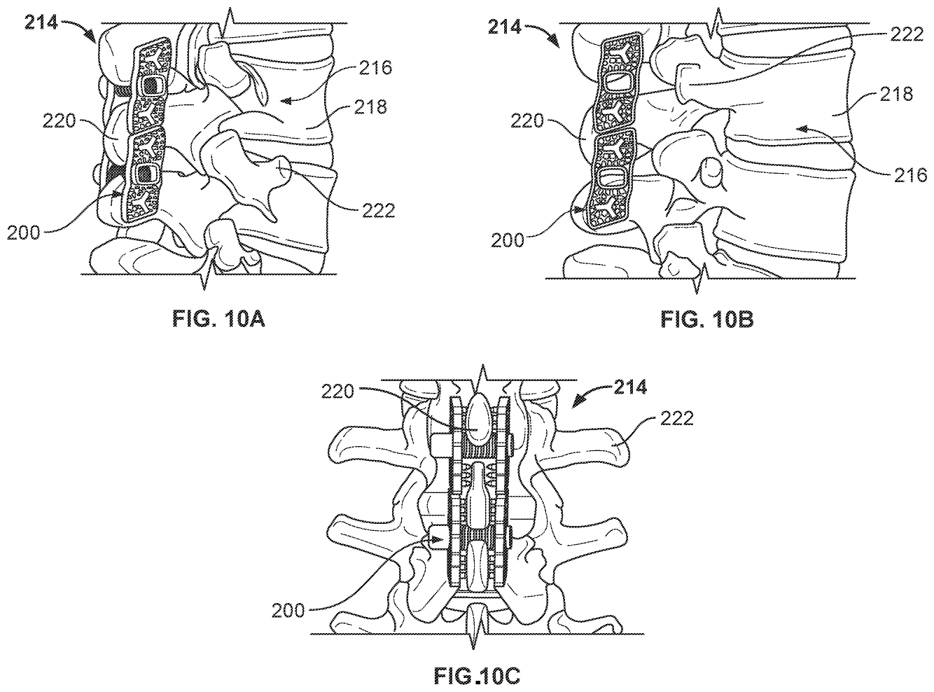

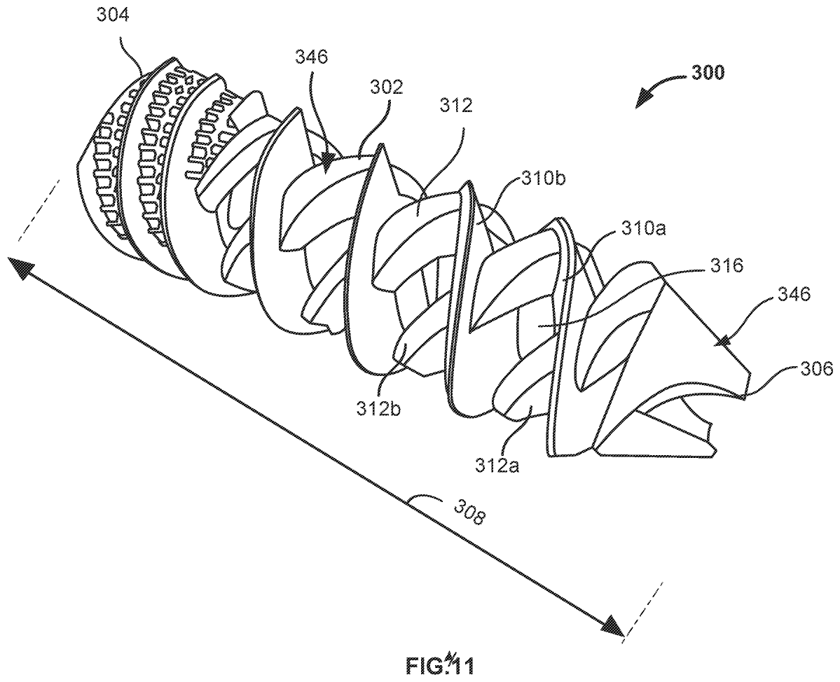

As shown in FIGS. 10A-10C, the ISP device 200 may be inserted into the spine 214 similarly to ISP device 100. The ISP device 200 may be inserted between at least two vertebrates 216 along the spine 214. The two vertebrates 216 may comprise vertebrae bodies 218, spinous processes 220, and transverse processes 222. In preferred embodiments, the ISP device 200 may be inserted between the spinous processes 220 of the vertebrates 216. The ISP device 200 may be secured in place by the midspring portion 206. The midspring portion 206 and spring coil 210 may be used to adjust the distance between the first side plate 202 and the second side plate 204. For example, the midspring portion 206 and spring coil 210 may be configured to narrow the distance between the first side plate 202 and the second side plate 204. Further, the midspring portion 206 and spring coil 210 may be configured to widen the distance between the first side plate 202 and the second side plate 204.

In some embodiments, one of at least the midspring portion 206 and spring coil 210 may be compressed or decompressed to adjust the distance between the first side plate 202 and the second side plate 204. For example, the midspring portion 206 and the spring coil 210 may be compressed to reduce the distance between the first side plate 202 and/or the second side plate 204. Further, the midspring portion 206 and the spring coil 210 may be relaxed, stretched, or in a decompressed state to widen the distance between the first side plate 202 and the second side plate 204. Generally, the distance between the first side plate 202 and the second side plate 204 may be adjusted until a desired distance between the first side plate 202 and the second side plate 204 is achieved and/or until the ISP device 200 is in a desired position and orientation in the spine 214. In some embodiments, the distance may be adjusted until first plate grip projections 208a and second plate grip projections 208b contact the spinous processes 220 of the vertebrates 216. The first plate grip projections 208a and the second plate grip projections 208b may contact the spinous processes 220 by engaging with the spinous processes 220, for example by physically touching a surface or sub-surface level of the spinous processes 220. In some embodiments, the first plate grip projections 208a and the second plate grip projections 208b may interdigitate with the spinous processes 220.

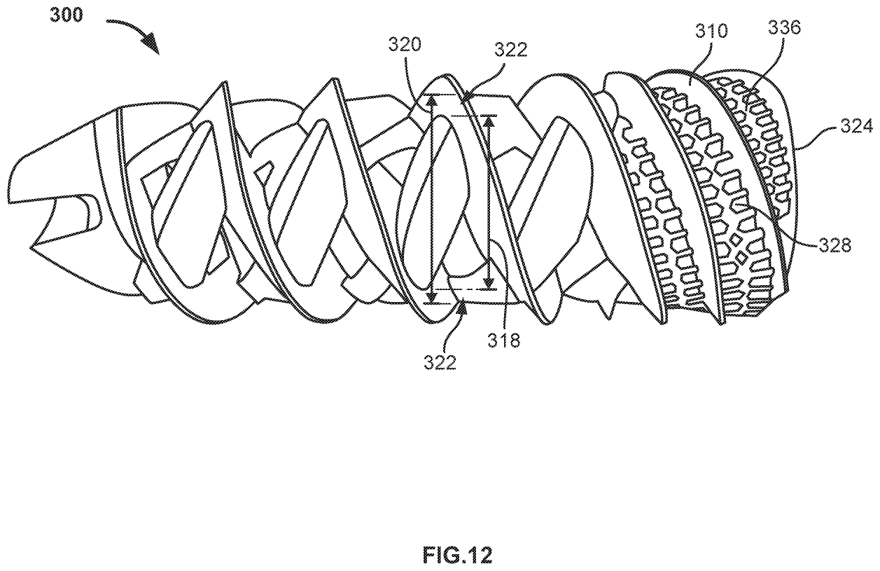

FIGS. 11-15 illustrates an embodiment of a bone screw 300. The bone screw 300 may comprise a body 302, a drive end 304, and a leading end 306. A distance between the drive end 304 and the leading end 306 may define a length 308. The bone screw 300 may further comprise threads 310. In various embodiments, the bone screw 300 may include at least one thread. In other embodiments, the bone screw 300 may include a plurality of threads 310. In one non-limiting example, the bone screw 300 may include at least or two threads 310, the two threads 310 may be disposed in a double helix relationship, as best shown in at least FIG. 11.

Bone screw 300 may further include an inner framework helix 312. In some embodiments, the inner framework helix 312 may be similar to the threads 310. The inner framework helix 312 may be about at least one element or two elements disposed in a double helix relationship. Both the threads 310 and the inner framework helix 312 may be any number of individual threads or members, but, in preferred embodiments, the bone screw 300 may include at least two threads 310 that may be offset substantially about 180 degrees on the circumference of the bone screw 300. Alternatively, the at least two threads 310 may be offset less than or more than about 180 degrees on the circumference of the bone screw 300.

The inner framework helix 312 may be orientated in an opposite direction of a rotation of the threads 310. The direction of rotation may be either a “right hand” helix and/or a “left hand” helix. In some embodiments, a first inner framework helix 312a and a second inner framework helix 312b may be arranged in a parallel relationship and may intersect a first thread 310a and a second thread 310b at a number of intersection joints 314. In some embodiments, a number of intersection joints 314 may be at least about one, two, three, four, five, and the like. In other embodiments, a bone screw 300 may have a plurality of intersection joints 314. The first inner framework helix 312a, the second inner framework helix 312b, the first thread 310a, and the second thread 310b may define one or more openings 316. In some embodiments, a bone screw 300 may have a plurality of openings. Further, the inner framework helix 312 may have a cross-sectional shape such as rectangular or conical. In one embodiment, inner framework helix 22 may have a rectangular shape. Alternatively, the inner framework helix 22 member may be circular, oval-shaped, or any other cross-sectional shape providing a desired stiffness and flexure.

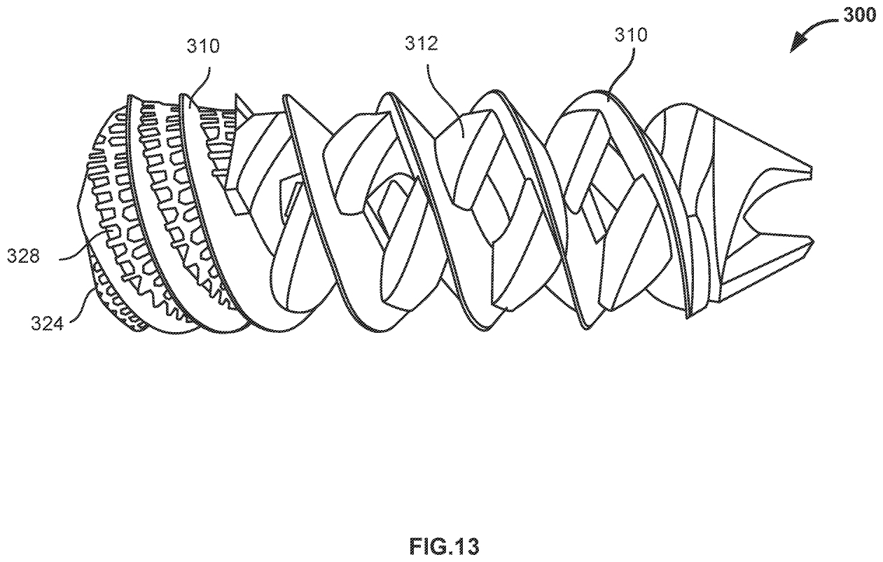

An inner diameter 318 of the threads 310 of the bone screw 300 may smaller than an outer diameter 320 of the inner framework helix 312, a best illustrated in FIG. 12. By varying the size of the inner diameter 318 of the threads 310 and the outer diameter 320 of the inner framework 312, an overlap 322 of volume may be created between the two helices where they may overlap, as bet shown in FIG. 13. This overlap 322 may vary in the percentage of overlap, but, in one embodiment, the overlap 322 may be about 50%. In other embodiments, the overlap 322 may be between about 45-55%, about 40-60%, about 25-75%, or about any other percentage of a thickness of the thread 310. In some embodiments, the dual helix bone screw 300 may be additively manufactured in Grade V titanium and by selective laser melting. Such manufacturing methods may create a rough surface that may allow for increased bone growth. Alternatively, the bone screw 300 may be manufactured by other processes such as casing, molding machining, joining, forging, and the like. Generally, the bone screw 300 may be additively manufactured with a biocompatible material. In some embodiments, the bone screw 300 may comprise a material of at least one of using polyethylethylketone (PEEK) plastic, nitinol metal or carbon fiber embedded additive manufacturing methods and the like.

Further, the driven end 314 of screw 312 may include a head portion 324 which may have a driver receptacle 326 disposed therein. In such embodiments, however, the head portion 324 may not differ in diameter from the remainder in the bone screw. Further, the threads 310 may extend to the driven end 314. As would be understood by one skilled in the art, such construction may be referred to as, “headless.” Moreover, the head portion 324 may include a porous construction such that head portion 324 may comprise one or more pores 328. The pores 328 may be configured substantially throughout the entire head portion 324. In some embodiments, the pores 328 may be configured throughout a portion or the entirety of he had portion 324. The pores 328 may be radially oriented, or, in some embodiments, the bone screw 300 may comprise a perforated outer wall 330 that may have a thickness 332 dimension substantially surrounding a cannula 334. In some embodiments, the perforations may be an open lattice structure in accordance with the present disclosure. Threads 310 may be positioned on top of or be integral with a perforated head wall 336.

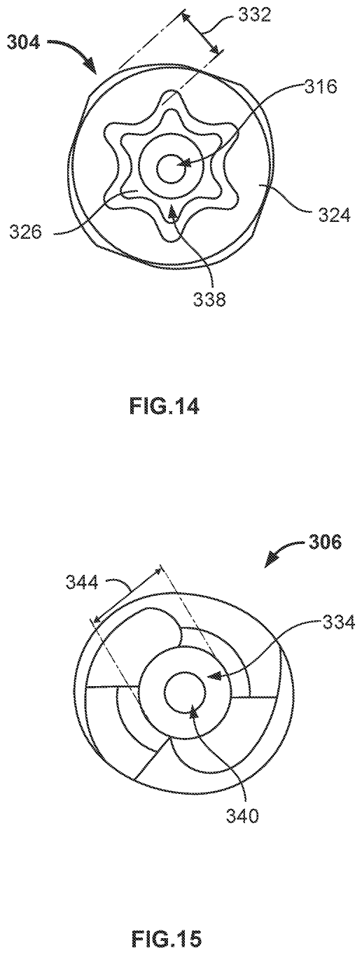

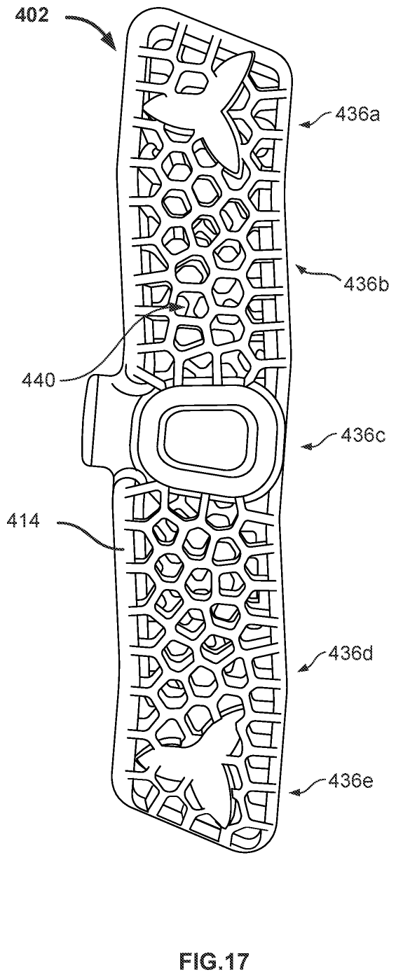

The inner framework helix 312 may define the cannula 334 through substantially the length 308 of the bone screw 310, as best illustrated in FIGS. 14 and 15. In some embodiments, the cannula 334 may be at least partially the length 308 of the bone screw 310. Preferably, the cannula is the entirety of the length 308 of the bone screw 310. The cannula 334 may include a driven end opening 338 and a leading end opening 340 that may define a length 342 dimension and a cannula diameter 344. In other embodiments, the cannula 334 may be present solely within a portion of the inner framework helix 312 with solid ends. One benefit of such embodiments may be that flexure in the axial direction in compression and/or tension is permitted, while the solid ends may be configured to provide rigidity when guides and/or a through-cannula are not required. As shown best in in FIG. 17, a desired driver receptacle 326 may be formed in the head portion 324. Further, the driver receptacle 326, in at least one embodiment, may be configured to receive a Torx driver head. However, any known driver, such as square, Phillips, hex, slot, or any other driver now known or hereafter developed may be utilized.

Elements of the bone screw 300 may have a rough surface 346 that may comprise a number of raised and depressed portions that may be on the inner framework helix 312. In some embodiments, a surface of the threads 310 may be relatively smoother than the surface of the inner framework helix 312. By having a relatively smooth surface, cutting through the bone may be relatively easier as the threads 310 engage a bone surface. Further, such surfaces may allow for better adhesion and on growth from the bone to the inner framework helix 312 and through the one or more openings 326. In other embodiments, a surface of the leading edge and/or leading end 306 may include a rough surface 346.

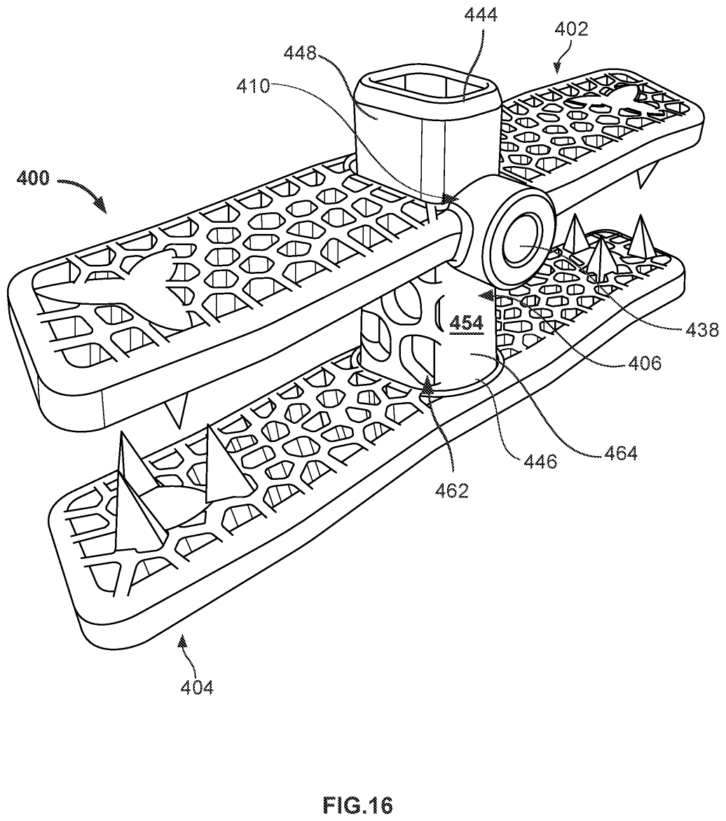

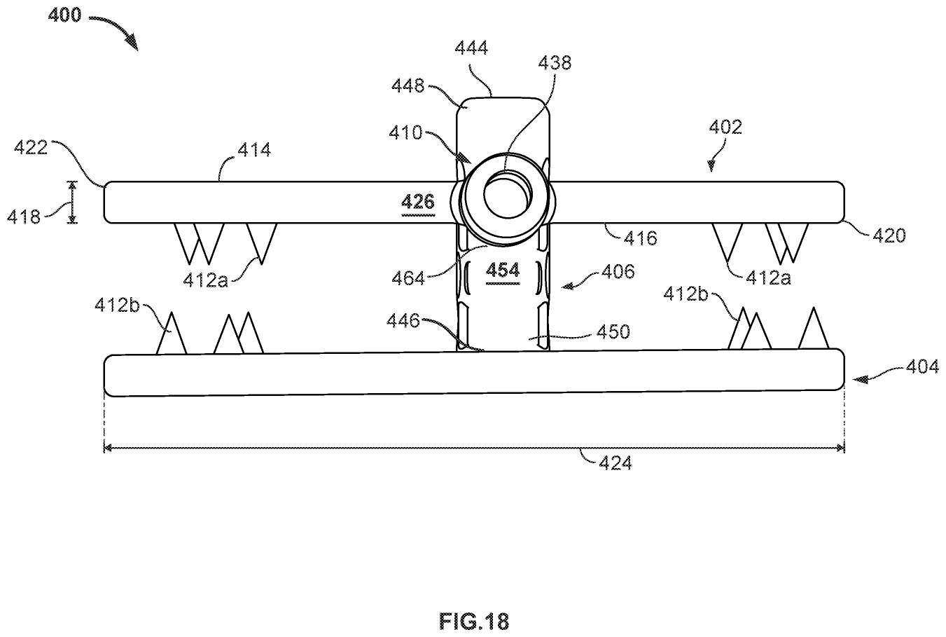

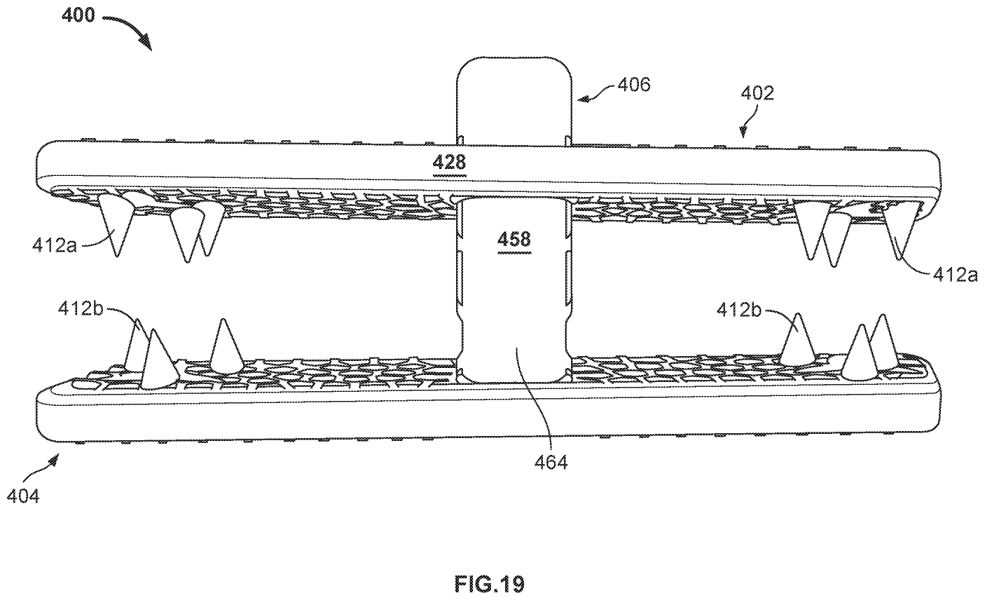

FIGS. 16-22 illustrate an embodiment of the ISP device 400 which may comprise a first side plate 402, a second side plate 404, and a transverse bridge member 406. Further, the ISP device 400 may comprise a guide channel 408, fixation member 410, and one or more grip projections 412. The shape of the first side plate 402 and the second side plate 404 may substantially be a rectangular prism or a plate shape. In alternative embodiments, the first side plate 402 and second side plate 404 may be another 3D shapes. The ISP device 400 may be substantially produced, manufactured, positioned, and/or oriented in accordance with the present disclosure and as described above. In various embodiments, the ISP device 400, and the components included therein, may be substantially similar to the ISP device 100 as previously described.

As best shown in FIGS. 16-19, the first side plate 402 may include a first side plate first side 410 and a first side plate second side 416. A distance between the first side plate first side 410 and the first side plate second side 416 may define a thickness 418 dimension. The first side plate 402 may include a first side plate first end 420 and a first side plate second end 422. A distance between the first side plate first end 420 and the first side plate second end 422 may define a length 424. Further, the first side plate 402 may include a first side plate top face 426 and a first side plate bottom face 428. A distance between the first side plate top face 426 and the first side plate bottom face 428 may define a width 430. The first side plate 402 may include a first side plate first end surface 432 and a first side plate second end surface 434. A distance between the first side plate first end surface 432 and the first side plate second end surface 434 may define a thickness 418 and/or a width 430.

Yet further, the first side plate 402 may also have one or more angular steps 436a-e along the first side plate top face 426 and the first side plate bottom face 428. In preferred embodiments, the angular step 436a-e may be between about 90-270 degrees. Alternatively, the angular steps 436a-e may be about less than or greater than 90-270 degrees. In other embodiments, the angular steps 436a-e may be between about 150-210 degrees. Alternatively, the angular steps 436a-e may be about less than or greater than about 150-210 degrees.

The first side plate second side 416 may include one or more grip projections 412a that protrude inwards towards the second side plate 404 and the transverse bridge member 406. The one or more grip projections 412a may be configured to contact and/or engage with the surrounding environment of the ISP device 400 such that the ISP device 400 may be positioned and maintained in place amongst other functionalities. Similar to other embodiments, the one or more grip projections 412a may have a 3D shape that is conical or pyramidal. Alternatively, the one or more grip projections 412a may have a shape that is angular, rounded, or a combination thereof. In exemplary embodiments, the one or more grip projections 412a may have a base end that extends from the first side plate second side 416 to a distal end. In one example, the grip projections 412a may have a conical shape with a rounded base end and a pointed tip (distal end). Further, the one or more grip projections 412a may protrude inwardly with respect to the second side plate 404 and the transverse bridge member 406. That is, the one or more grip projections 412a may protrude towards the second side plate 404 and the transverse bridge member 406. In preferred embodiments, the first side plate second side 416 may include one or more grip projections 412a (for example, about three, about four, about five, and the like) on each side of the transverse bridge member 406.

In one embodiment, three grip projections 412a are in a group on one side of the transverse bridge member 406, and another three grip projections 412a are in a group on another side of the transverse bridge member 406. Alternatively, the number of grip projections 412a on either side of the transverse bridge member 106 may be different such that there are more grip projections 412a on one side of the transverse bridge member 406 than the other side.

The first side plate 402 may also include a fixation member 410. The fixation member may include a set screw 438. The set screw 438 may be configured to receive a Torx driver head. Alternatively, other drivers such as square, Phillips, hex, slot, or any other driver now known or hereafter developed may be utilized. In preferred embodiments, a distance between the first side plate 402 and the second side plate 404 may be adjusted until a desirable distance is reached, and the fixation member 410 and/or the set screw 438 may be used to position and secure the ISP device 400 in place. For example, a distance between the first side plate 402 and the second side plate 404 may be narrowed or widened by using the fixation member 410 and/or the set screw 438.

Moreover, the ISP device may include one or more porous members 438. The first side plate 402 may include a first side plate porous member 440a, which may substantially comprise a majority of the first side plate 402. Alternatively, the first side plate porous member 440 may only substantially partially include the first side plate 402. The first side plate porous member 440a may include an open lattice structure to create openings 442 for contiguous bone growth through or into the first and second side plates 402 and 404. In various embodiments, the second side plate 404 may have a similar construction to the first side plate 402. For example, the second side plate 404 may include grip projections 412b. In preferred embodiments, the second side plate 404 and first side plate 402 may be substantially similar.

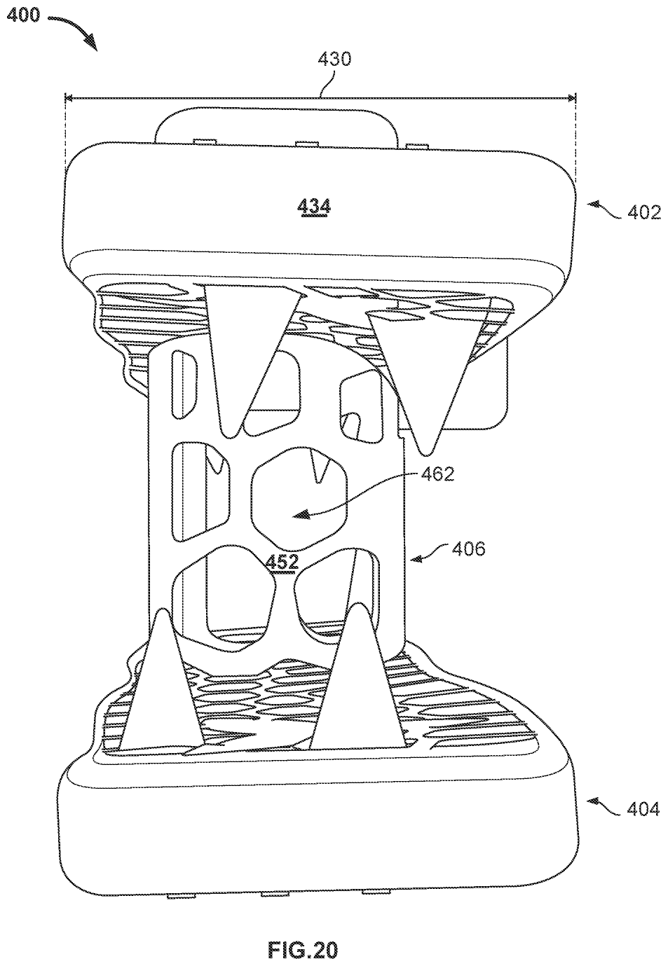

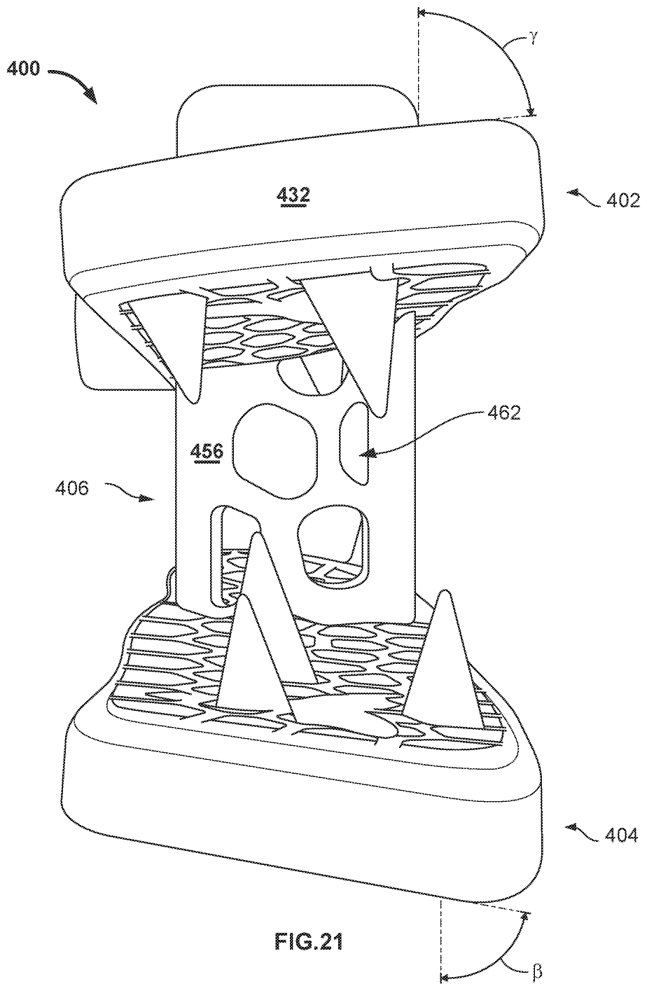

The transverse bridge member 406 may include a transverse bridge member first end 444 and a transverse bridge member second end 446. The transverse bridge member first end 444 may include a first end solid portion 448, and the transverse bridge member second end 446 further includes a second end solid portion 450. The transverse bridge member 406 may comprise a first side 452, a second side 454, a third side 456, and a fourth side 458. In some embodiments, the first side 452, the second side 454, the third side 456, and the fourth side 458 may be connected by curved side transitions 460 to form a substantially cylindrical guide channel 408. In other embodiments, the guide channel 408 may have a tubular shape with rounded transitions. In alternative embodiments, the guide channel 408 be another 3D shapes which may be angular, rounded, or a combination thereof. As shown best in FIGS. 20 and 21, the transverse bridge member 406 may include one or more relatively larger pores or holes 462 through at least one or more of the first side 452, the second side 454, the third side 456, and/or the fourth side 458. The one or more holes 462 may be designed as a generally open and porous structure. Further, the holes 462 may reduce weight, allow for bone through growth, and/or provide some flexure when the ISP device 500 is in operation, amongst other functions. In a preferred embodiment, the first side 452 and the third side 456 may include holes 462, and the second side 454 and the fourth side 458 include a solid wall 464.

In accordance with the present disclosure, the transverse bridge member 406 may have a width and a longitudinal axis perpendicular to the width of the transverse bridge member 406. The first and second side plates 402 and 404 may be parallel and disposed on opposite ends of the transverse bridge member 106 along the longitudinal axis. The first side plate 402 may be disposed near the transverse bridge member first end 444 of the transverse bridge member 406, and the second side plate 404 may be disposed at the transverse bridge member second end 446. As shown best in FIGS. 20 and 21, the first side plate 402 may be oriented relative to the transverse bridge member 406 and the longitudinal axis at an angle γ, and the second side plate 404 may be oriented relative to the transverse bridge member 406 and the longitudinal axis at an angle β. Angles γ and β may be less than or greater than about 90 degrees. In preferred embodiments, angles γ and β may be between about 60 and 90 degrees.

The distance between the first side plate 402 and the second side plate 404 may be adjusted via the fixation member 410. For example, the fixation member 410 may be configured to move the first side plate 402 and/or the second side plate 404 closer or farther away from one another along the transverse bridge member 406. Moreover, the fixation member 410 may be configured to maintain each the first and second side plates 402 and 404 at a desired, respective angle.

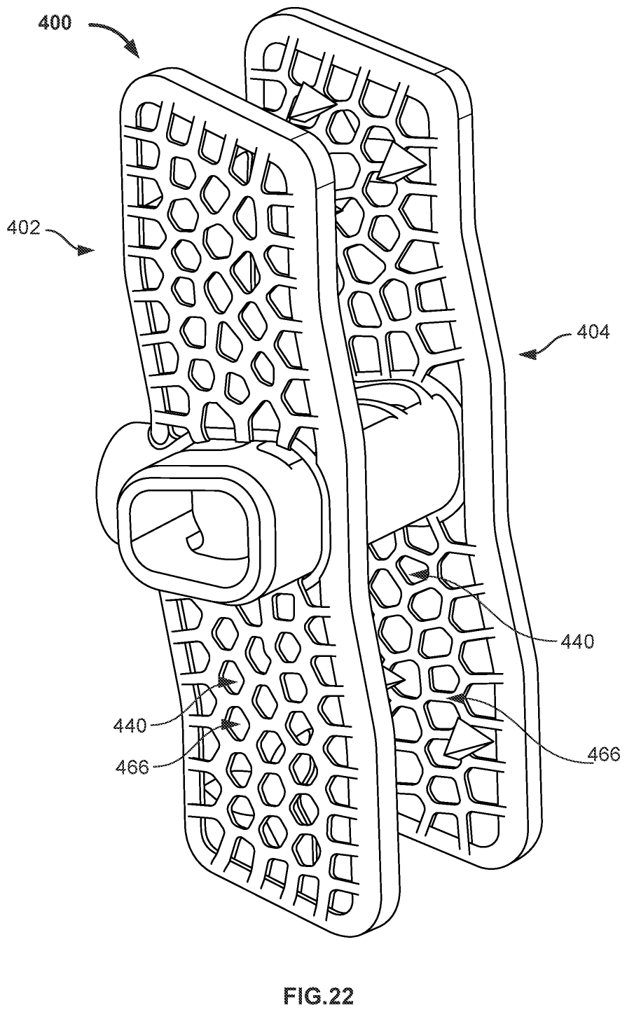

Turning to FIG. 22, an embodiment of ISP device 400 including a bone graft material 466 is provided. The porous member 440, which comprises a majority of the first and second side plates 402 and 404, may be filled with a bone graft material. In some embodiments, the porous members 438 may improve contiguous bone growth through or into the first and second plates 402 and 404.

From the foregoing, it will be seen that this invention is one well adapted to attain all the ends and objects hereinabove set forth together with other advantages which are obvious, and which are inherent to the structure. It will be understood that certain features and sub combinations are of utility and may be employed without reference to other features and sub combinations. This is contemplated by and is within the scope of the claims. Since many possible embodiments of the invention may be made without departing from the scope thereof, it is also to be understood that all matters herein set forth or shown in the accompanying drawings are to be interpreted as illustrative and not limiting.

The constructions and methods described above and illustrated in the drawings are presented by way of example only and are not intended to limit the concepts and principles of the present invention. Thus, there has been shown and described several embodiments of a novel invention.

As is evident from the foregoing description, certain aspects of the present invention are not limited by the particular details of the examples illustrated herein, and it is therefore contemplated that other modifications and applications, or equivalents thereof, will occur to those skilled in the art. The terms “having” and “including”, and similar terms as used in the foregoing specification are used in the sense of “optional” or “may include” and not as “required”. Many changes, modifications, variations and other uses and applications of the present construction will, however, become apparent to those skilled in the art after considering the specification and the accompanying drawings. All such changes, modifications, variations and other uses and applications which do not depart from the spirit and scope of the invention are deemed to be covered by the invention which is limited only by the claims which follow.

We claim:

1. A spinous process fixation device comprising:
a transverse bridge member having a width and a longitudinal axis perpendicular to the width;
a first side plate positioned at about a first end of the transverse bridge member, the first side plate comprising at least one first grip projection that protrudes towards the transverse bridge member; and
a second side plate positioned at about a second end of the transverse bridge member, the second side plate comprising at least one second grip projection that protrudes towards the transverse bridge member.

2. The spinous process fixation device of claim 1 further comprising a fixation member and a screw configured to adjust a distance between the first side plate and the second side plate.

3. The spinous process fixation device of claim 1 further comprising a guide channel.

4. The spinous process fixation device of claim 1, wherein at least one of the transverse bridge member, the first side plate, and the second side plate include a plurality of open lattice structures.

5. The spinous process fixation device of claim 4, wherein the plurality of open lattice structures defines a plurality of openings, and wherein the openings are designed to promote bone growth through the spinous process fixation device in more than one direction.

6. The spinous process fixation device of claim 5, wherein the open lattice structures and the defined openings are configured for packing a bone graft material.

7. The spinous process fixation device of claim 5, wherein a plurality of openings about the transverse bridge member are larger than a plurality of openings about the first side plate and the second side plate.

8. The spinous process fixation device of claim 1, wherein the first side plate and the second side plate are oriented perpendicular with respect to the longitudinal axis, and wherein the first side plate and the second side plate are oriented parallel with respect to each other.

9. The spinous process fixation device of claim 1, wherein the first side plate and the second side plate are each oriented at an angle with respect to the longitudinal axis.

10. The spinous process fixation device of claim 1, wherein the at least one first grip projections and the at least one second grip projections are configured to contact and engage the installation surrounding environment to maintain position of the first side plate, the second side plate, and the transverse bridge member.

11. A spinous process fixation device comprising:
a spring member having a diameter and a longitudinal axis in a direction perpendicular to the diameter;
a first side plate positioned at about a first end of the spring member, the first side plate comprising at least one first grip projection that protrudes towards the spring member; and
a second side plate positioned at about a second end of the spring member, the second side plate comprising at least one second grip projection that protrudes towards the spring member.

12. The spinous process fixation device of claim 11, wherein the first side plate and the second side plate are oriented perpendicular with respect to the longitudinal axis, and wherein the first side plate and the second side plate are oriented parallel with respect to each other.

13. The spinous process fixation device of claim 11, wherein the spring member comprises a spring coil, and wherein the spring member is configured to compress or decompress to adjust a distance between the first side plate and the second side plate.

14. The spinous process fixation device of claim 11, wherein at least one of the first side plate and the second side plate each comprise a solid member.

15. The spinous process fixation device of claim 11, wherein the spring member is configured to limit lateral flexure.

16. The spinous process fixation device of claim 11, the spinous process fixation device comprising a Grade V titanium material.

17. A surgical screw comprising:
at least one helical thread arranged in one of a right-hand helix or a left-hand helix, said at least one helical thread having a first thickness partially defined by an inner diameter; and
at least one inner framework helix arranged in the other of the right-hand helix or the left-hand helix, said at least one inner framework helix having a second thickness partially defined by an outer diameter,
wherein the inner diameter of the at least one helical thread is smaller than the outer diameter of the at least one inner framework helix thereby creating an overlap of the first thickness and the second thickness.

18. The surgical screw of claim 17, wherein the overlap is 25-75% of the first thickness of the at least one helical thread.

19. The surgical screw of claim 17, wherein a surface of the at least one inner framework helix is rough.

20. The surgical screw of claim 17, wherein a surface of the at least one helical thread is smooth.
